(19) United States
(12) Patent Application Publication
(10) Pub. No.: US 2011/0183427 A1
(43) Pub. Date: Jul. 28, 2011

(54) METHOD FOR SEPARATING THE CONSTITUENTS OF A COMPLEX MIXTURE OF PROTEINS TO SUBMIT TO PROTEOMIC ANALYSIS AND APPARATUS THEREFOR

(75) Inventors: Lorenzo Citti, Lucca (IT); Silvia Rocchiccioli, Pisa (IT); Alberto Mercatanti, Pisa (IT); Claudia Boccardi, Livorno (IT); Oberdan Parodi, Viareggio (IT)

(73) Assignee: CNR-CONSIGLIO NAZIONALE DELLE RICERCHE, Roma (IT)

(21) Appl. No.: 13/121,438

(22) PCT Filed: Sep. 29, 2009
(86) PCT No.: PCT/IB2009/006989

§ 371 Date: Apr. 5, 2011

(30) Foreign Application Priority Data
Sep. 29, 2008 (IT) PI2008A000102

(51) Int. Cl. G01N 33/50; B01D 21/00; G01N 21/00

(52) U.S. Cl. 436/86; 422/527; 422/82.05

(57) ABSTRACT

A method and an apparatus for separating the constituents of a complex mixture of proteins, for example an organic fluid, such as blood plasma, to submit to proteomic analysis. The method comprises, in particular, a step of distributing the complex mixture of proteins on a determined number n of separation elements different from each other (101). Each separation element separates a determined group of proteins from the other proteins present in the complex mixture (102) according to determined criteria. The proteins thus isolated from each separation element are diluted and neutralised (103) for being then sent to an analytic instrument, for example a column for high resolution liquid chromatography, or HPLC (104b), and then a spectrometer of MS type, or of MS/MS type. Each separation element has particular chemical-physical features that capture by adsorption only the proteins belonging to a determined group, i.e. having similar features.

### PLAYING FIELD OF THE INVENTION

The present invention relates to an automatic system that is integrated in a single device for separating the constituents of a complex mixture of proteins to analyse with “proteomic” techniques.

### BRIEF DESCRIPTION OF THE PRIOR ART

In the prior art the manipulation of biological samples for analytic purposes represents a technical constraint that is historically recurrent and that can deeply influence the relationship between an analyte and the method for its measurement.

Normally, this manipulation determines a plurality of procedures which, in case of a mixture of proteins, require consolidated biochemical skills, various types of instruments, long execution time, and give results that are frequently variable responsive to different operators and laboratories. In this context the “proteomic” methodologies are presently accomplished.

The “proteomic” methodologies, in particular, have the goal to analyse and recognize the various proteins that are contained in a biological sample by means of sophisticated mass spectrometry techniques. This type of “landscape” analysis is particularly useful for studies on the cause that are at the basis of pathological processes, for searching the markers of a disease, for analysing food products, for the pharmaceutics industry etc. However, the huge complexity of a mixture of proteins needs simplifications that are necessary in order to apply this type of technology.

Among the techniques that are used there are the two-dimensional electrophoretic separation (2D-GEL electrophoresis) or the multiple chromatography, adopted to obtain fractioning of the proteins that are contained in the biological samples and to provide sub-populations that are more restricted and require a much easier analysis. They are technologies that are sophisticated, slow and sometimes have a low reproducibility. It follows that identical samples that are analysed by different laboratories provide different results that sometimes are partial and can overlap only partially as well as they can considered only contiguous results that are complementary to each other.

Normally, the different strategies, which are adopted for separation of proteins that are contained in a complex mixture deriving from biological samples, generate sub-populations of proteins that are subsets defined by chemical and physic conditions and that depend on the used fractioning method. Thus, whereas the two-dimensional electrophoretic method separates the proteins responsive to their isoelectric point and of the molecular size, the liquid chromatography with reverse-phase separates the proteins responsive to their different hydrophobic features. Therefore, the use of two different fractioning methods causes the separation of different populations of proteins from each other.

More in detail, the two-dimensional electrophoresis, or 2D electrophoresis, is a type of electrophoretic technique that is used in proteomics for separating proteins in a complex protein mixture, i.e. consisting of several different protein species.

For carrying out the separation of the different proteins 2D electrophoresis exploits two “orthogonal” dimensions, i.e. two dimensions in which the separation is carried out by exploiting two different physical principles and precisely the isoelectric point and the molecular weight.

The first dimension usually considers a pH gradient obtained by amphoteric molecules that are caused to migrate on a support, which is made up generally by a gel of polyacrylamide and is put in an electric field. Also exist portions, or “strips”, of prefabricated structures, in which are immobilized of the ampholytes. During polymerization, the gel of a strip is subject to an external electric field in order to generate the pH gradient.

In the second dimension the sample is treated with sodium dodecylsulphate (SDS) in order to confer to the proteins an electric net negative charge. Then, a SDS-PAGE step follows where the protein species are sorted responsive to their molecular weight. The proteins are eventually shown by different colouring, using for example Blue of Coomassie, or silver nitrate

Such procedure is, however, slow and difficult to carry out, and requires the use of many operators that intervene in the different steps. This excludes the possibility to process many samples contemporaneously and of working them in an automatic way.

Furthermore, the 2D electrophoresis allows measuring only the constituents that are present in the mixture in an elevated amount, whereas those present in a reduced amount are not measured.

Examples of 2D electrophoresis are, for example, described in US2005037353, US2004259221 and in US2002153252.

A different technique of separation is disclosed in US20040224362. In particular, the method provides the introduction of a sample solution which contains the analyte in an extraction channel, or capillary. The analyte is adsorbed on an extraction surface that covers the surface of the extraction channel. The adsorbed analyte is then eluted in a desorbing solution.

Also in this case the described technique has some drawbacks.

Firstly, the process described allows to process only limited amounts of the samples. In fact, since the separation occurs on an extraction surface, it is necessary that the dimension of the extraction capillaries is extremely reduced, i.e. in a micro-range. Therefore, it is not possible to treat a sample that is present in a determined extraction capillary by a succession of operations. The amount of the sample that is obtained at the end of the first step is, in fact, isofficient in order to obtain significant results in a successive step.

In the document a particular embodiment is described that provides a determined number of extraction capillaries. At the same time into the extraction capillaries either determined amounts of a same sample can be put, or more operations can be carried out on determined amounts of different samples.

In both cases the samples put in the extraction capillaries can be processed at the same time by treating them in a same operation. In order to treat the samples in a differentiated way it is, instead, necessary to execute a succession of operations, or “steps”. More precisely, at each “step” of the succession a single operation is carried out.

For the above reasons, it is not possible to treat in a single pass at the same time the samples processed in a way that is differentiated for separating the various constituents of the mixture.

Therefore, also the technique described in document US20040224362 is not much flexible and causes relevant loss of time.

### SUMMARY OF THE INVENTION

It is therefore a feature of the present invention to provide an apparatus for separating the constituents of a complex mixture of proteins for proteomic analysis that is automatic and integrated, and also capable of obtaining, in a rapid, automatic and reproducible way, as many varieties as possible of sub-populations of proteins starting from a complex biological sample.

It is also a feature of the present invention to provide such an apparatus that adapts itself to the analytic needs of the user.

It is another feature of the present invention to provide such an apparatus that allows a modular expansion in accordance with the geometries that are typical robotic apparatus for laboratories, in order to achieve a continuous updating on the basis of new products that are provided by molecular technologies (FAB, aptamer, etc.).

It is a further feature of the present invention to provide such an apparatus that allows a completely automatic manipulation of the sample in order eliminate human artifacts.

It is also a feature of the present invention to provide a method for separating the constituents of a complex mixture of proteins for proteomic analysis that allows a multifunctional integration of different separation devices of the protein fractions on the basis of chemical and physical parameters capable of providing a wider range and diversified of proteic sub-populations.

These and other features are accomplished with one exemplary apparatus, according to the invention, for separating the constituents of at least one complex mixture of proteins to submit to proteomic analysis, comprising:
a plurality of separation elements, said separation elements of said plurality having chemical-physical features such that they can capture selectively by adsorption proteins belonging to a determined homogeneous group of proteins of said complex mixture;
a holding means, for holding said plurality of separation elements according to a determined array,
a distribution means adapted to distribute said complex mixture of proteins on each separation element of said plurality of separation elements;
an application means on each separation element for applying a determined solvent that can cause the removal of a respective group of proteins from the respective separation element obtaining respective resulting solutions;
a collection means, for collecting said respective resulting solutions for a following analysis in order to identify the single proteins of each group.

Preferably, the separation elements are columns that are packed with a packing material, said packing material permeated, in use, by the complex mixture of proteins and by the solvent.

In an exemplary embodiment of the invention, each packed column comprises a packing material of different nature from that of the other packed columns.

In particular, the packing material can be selected from the group comprised of:
chemically deactivated silica;
a porous polymer;
a nonporous polymer.

In particular, the means for collecting can comprise at least one plate having microwells, or “microwell plate”.

Advantageously, the distribution means and/or the application means comprise multichannel pipettes.

In particular, the distribution means can be operatively connected to a program means, said program means adapted to operate said distribution means so that they distribute a determined amount of the complex mixture into determined separation elements.

Similarly, the application means can be operatively connected to a program means, said program means adapted to operate said application means so that they apply determined amount of solvent into determined separation elements.

For example, the application means and/or the distribution means can be robotized systems of commercial type such as Beckman robotized systems.

Advantageously, said separation elements comprises at least two of the following separation elements:
a strong anionic exchange separation element (SAX) that is adapted to separate acid proteins;
a strong cationic exchange separation element (SCX) that is adapted to separate basic proteins;
a reverse-phase separation element (RP-18) that is adapted to separate hydrophobic proteins;
a separation element based on chelant metals (IMAC), or on titanium dioxide, or on other substances, which is adapted to separate phosphorylated proteins;
a separation element based on Lectin, in particular Concanavalin-A, that is adapted to separate glycosylated proteins;
a separation element based on an antibody having specific affinity with a determined group of proteins, said separation element adapted to separate said group of proteins having said specific affinity with said antibody;
a separation element based on an aptamer having specific affinity with a determined group of proteins, said separation element adapted to separate said group of proteins having said specific affinity with said aptamer;
or a combination thereof.

The separation elements described show examples that do not exclude other separation elements that can usefully be used for the purposes described in the present invention.

In particular, each said separation element captures by adsorption proteins belonging to a homogeneous group of proteins of particular analytic use, said homogeneous group of proteins selected from the group comprised of:
a group of acid proteins,
a group of basic proteins,
a group of hydrophobic proteins,
a group of phosphorylated proteins,
a group of glycosylated proteins,
a group of proteins having specific affinity with a determined antibody;
a group of proteins having specific affinity with a determined aptamer.

Advantageously, the means for collecting comprises containers for said resulting solutions which at least one among:
a container for acid proteins;
a container for basic proteins;
a container for hydrophobic proteins;
a container for phosphorylated proteins;
a container for glycosylated proteins;
a container for a homogeneous group of proteins having specific affinity with a determined antibody;
a container for a homogeneous group of proteins having specific affinity with a determined aptamer;
or a combination thereof.

In particular, the complex mixture of proteins to examine is comprised in an organic fluid, for example blood plasma.

Furthermore, means can be provided for collecting the non-adsorbed proteins on the corresponding separation element.

Advantageously, each separation element comprises a first zone, or inlet zone, and a second zone, or outlet zone, said distribution means and said application means acting at said first zone, said collection means for collecting the non-adsorbed proteins acting at said second zone.

In particular, between the first and the second zone of the separation element a determined packing material can be present.

Preferably, at least a first and a second separation element are provided that are adapted to separate by adsorption, i.e. to isolate, a same group of proteins starting from a same complex mixture of proteins. This way, a same group of proteins is isolated from the starting mixture and it is therefore possible to have two samples on which the predetermined measurement can be carried out to obtain a first test on the exactness of the results.

Advantageously, furthermore, a means is provided for analysing said respective resulting solutions that is adapted to identify the single proteins of each group.

In particular, the means for analysing said resulting solutions can comprise a chromatograph for liquid chromatography at high resolution (HPLC) and a mass spectrometer, the latter selected from the group comprised of: a mass spectrometer of MS type, or a mass spectrometer of MS/MS type.

In particular, before a step of analysis, to the sample to analyse a measured amount of a known specific proteolytic enzyme is added, for example trypsin, capable of breaking selectively some peptide bonds and to allow the analysis of the fragments resulting from said means for analyse.

Advantageously, the support means comprises a support having a plurality of through channels, in each through channel of said plurality of through channels a separation element of said plurality of separation elements being arranged.

In particular, the first inlet zone and the second outlet zone can be defined in each through channel through the respective separation element.

Advantageously, the solvent is a buffer solution containing a desorbing agent that is adapted to induce variation of pH, variation of ionic force, variation of polarity or other chemical-physical characteristic.

In an exemplary embodiment, the solvent can be a buffer solution containing a soluble agent that is capable of competing with the adsorbing elements of the packing material of the separation element.

In a further exemplary embodiment as solvent a buffer solution containing a denaturing agent can be used.

According to another aspect of the invention, a method for separating the constituents of a complex mixture of proteins to submit to proteomic analysis, comprises the steps of:
arranging a plurality of separation elements according to a determined array, said separation elements adapted to separate selectively by adsorption a determined homogeneous group of proteins from the other proteins present in the complex mixture of proteins;
distributing at least one complex mixture of proteins on each separation element of said plurality of separation elements;
selectively separating by each separation element a determined group of proteins from the other proteins present in the complex mixture;
applying a determined solvent on each separation element, said solvent adapted to remove each group of proteins from the respective separation element obtaining respective resulting solutions;
obtaining said resulting solutions from each separation element;
sending the resulting solutions to a step of analysis in order to identify the single proteins of each group.

Advantageously, before the step of applying said determined solvent on said separation element a step is provided of washing each separation element. The washing step is adapted to remove possible interfering molecules that have been co-adsorbed along with each group of proteins on the separation element.

In particular, a preliminary step can be provided of filtering a sample of organic fluid comprising the mixture of proteins. This filtering step that is adapted to separate the mixture of proteins from other substances present in the fluid sample which are removed.

Advantageously, before the step of sending the proteins to analysis a step is provided of dilution and neutralization of the resulting solutions.

### BRIEF DESCRIPTION OF THE DRAWINGS

The invention will be made clearer with the following description of an exemplary embodiment thereof, exemplifying but not limitative, with reference to the attached drawings in which:

FIG. 1 shows a block diagram of the main phases of the method according to the invention, for separating the constituents of a complex mixture of proteins to submit to proteomic analysis;

FIGS. 2 to 4 show diagrammatically a container used for executing the method according to the invention, in three different steps;

FIG. 5 shows a top plan view of a support plate that is adapted to house a determined number of containers similar to those shown in FIGS. 2 to 4;

FIGS. 6 and 7 show the support plate of FIG. 5 in a cross section according to arrows VI-VI in two different steps of the method;

FIG. 8 shows diagrammatically a perspective view of a possible exemplary embodiment of a support plate of the containers of FIG. 5;

FIG. 9 shows a perspective view of an apparatus, according to the invention, for separating the constituents of a complex mixture of proteins to submit to proteomic analysis.

### DETAILED DESCRIPTION OF SOME EXEMPLARY EMBODIMENTS

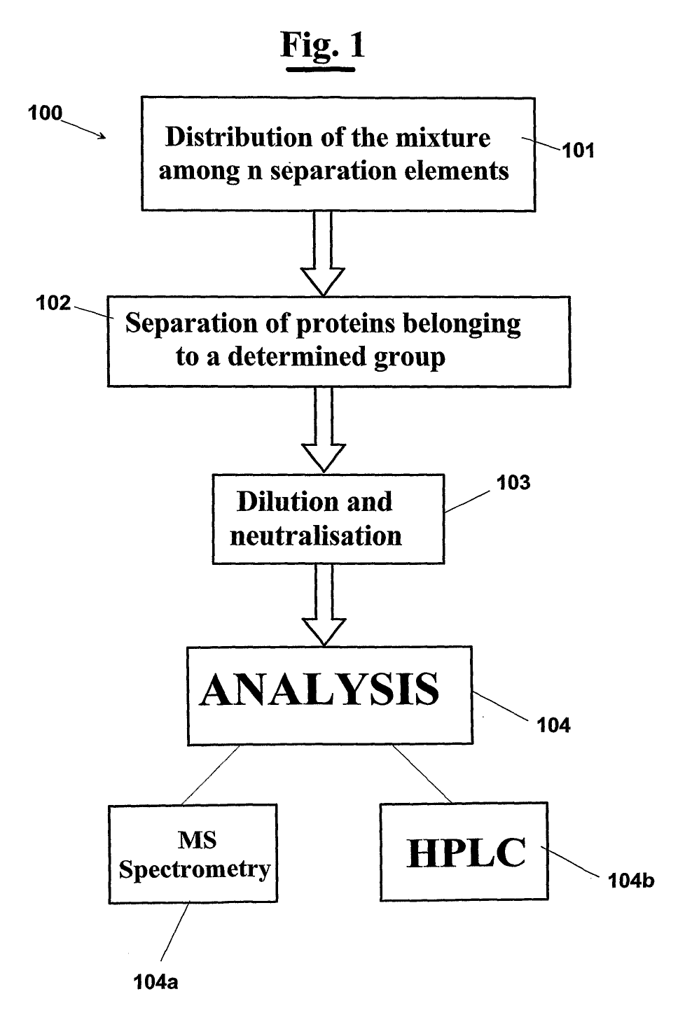

In FIG. 1 shows a block diagram 100 of the main phases of the method according to the invention, for separating the constituents of a complex mixture of proteins, for example an organic fluid, such as blood plasma, to submit to proteomic analysis.

Such method comprises, in particular, a step of distributing the complex mixture of proteins on a determined number n of separation elements that are arranged according to respective containers, block 101.

Then, each separation element separates a determined group of proteins from the other proteins present in the complex mixture, block 102.

The proteins thus isolated from each separation element are diluted and neutralised, block 103 for being then sent to an analysis instrument, block 104, for example a column for high resolution liquid chromatography, or HPLC (High Performance Liquid Chromatography), block 104b, and then a spectrometer of MS type, or of MS/MS type.

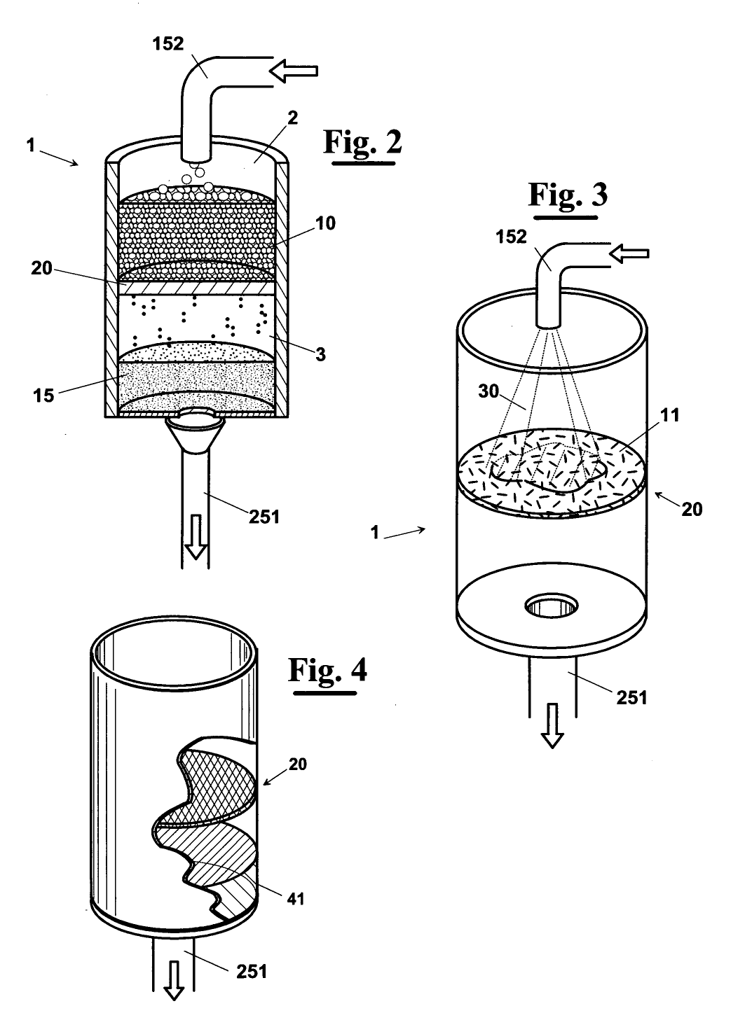

In particular, as diagrammatically shown in FIG. 2, each separation element 20 divides the corresponding container 1 into a first zone 2 and a second zone 3. More in detail, each separation element 20, for example a packed column, comprises a packing material with determined chemical-physical features such that it captures by adsorption only the proteins belonging to a determined group, i.e. having similar features, whereas its lets pass into the second zone 3 the other proteins present in the organic sample fluid which are removed from container 1 by a duct 251. The packing material can be chemically deactivated silica, a porous polymer, or a nonporous polymer.

The proteins 11 belonging to a determined unit and adsorbed on the separation element 20 are, then removed by applying determined solvents 30 that desorb the proteins 11 obtaining respective resulting solutions 41 that pass to the lower zone 3 of each container 1, from where they are collected in order to be sent to the steps successive. The solvents can be buffer solutions containing a desorbing agent that is adapted to induce an effect selected from the group comprised of: a variation of pH, a variation of ionic force, a variation of polarity. In an exemplary embodiment, the solvent can be a buffer solution containing a soluble agent that is capable of competing with the adsorbing elements of the packing material of the separation element. In a further exemplary embodiment the solvent can be a buffer solution containing a denaturing agent.

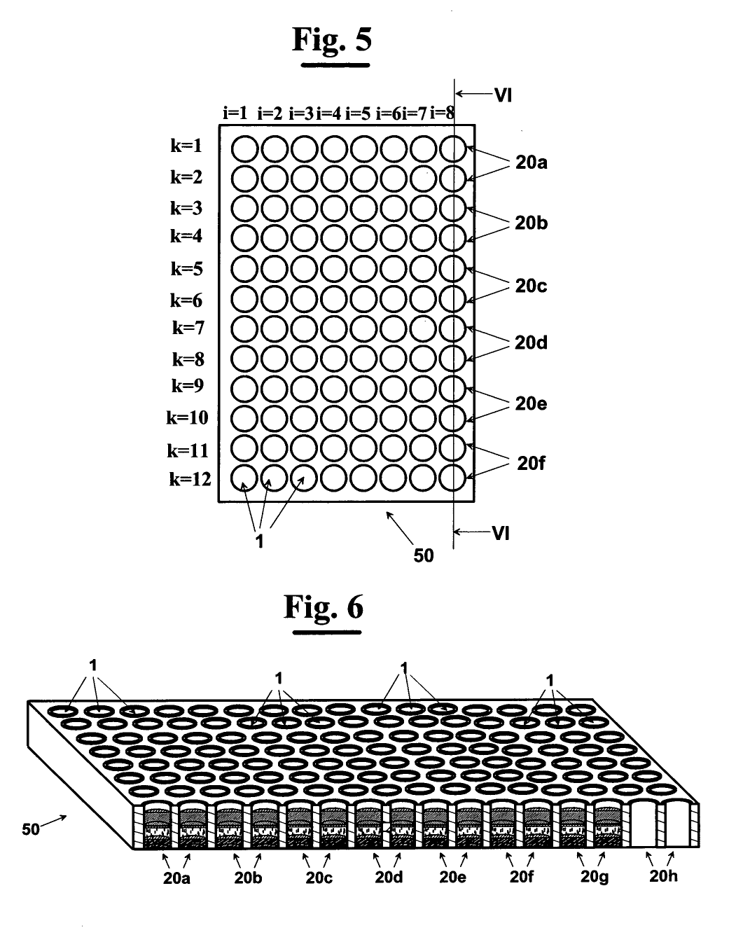

As shown in FIGS. 5 and 6, for example, a support plate can be provided comprising a determined number of containers 1 arranged on lines i and columns k, for example 12 lines and 8 columns for an overall number of 96 containers. The containers 1 arranged on a same line k have separation elements 20 all with the same features, so that they capture a same group of proteins. The containers 1 belonging at a same column i, instead, are loaded with a same sample of organic fluid. This way, an “array” of containers is obtained that can be processed at the same time in order to analyse a high number of different samples detecting for each of them the amount of the different proteins present. This allows obtaining an apparatus capable of automatizing the overall process obtaining a high saving concerning time, costs and reproducibility with respect to the known systems.

The separation elements 20 that are arranged on the same line are capable of adsorbing proteins having similar features, i.e. belonging to a same group.

In the example of FIG. 5, for each sample of organic fluid that is processed two separation elements are provided 20a capable of causing the adsorption of acid proteins from means of a strong anionic exchange (SAX), two separation elements 20b capable of adsorbing basic proteins from means of a strong cationic exchange (SCX), two separation elements 20c with reverse-phase (RP-18) capable of adsorbing hydrophobic proteins, two separation elements 20d based on chelant metals (IMAC) or on titanium dioxide or on other substances capable of adsorbing phosphorylated proteins, two separation elements 20e based on Lectin, or on Concanavalin-A, capable of keep glycosylated proteins, and alternatively, two elements 20f based on a determined antibody capable of capturing proteins of defined features, or two elements 20g based on a determined aptamer capable of keep proteins of defined features. Other separation elements, which discriminate the proteins on the basis of further features, can usefully be used for the purpose described in the present invention.

Once separated a determined group of proteins from the other proteins that are present in the complex mixture, in each container 1a specific solvent is added capable for removing the proteins adsorbed on the separation elements 20a-20g (FIG. 3).

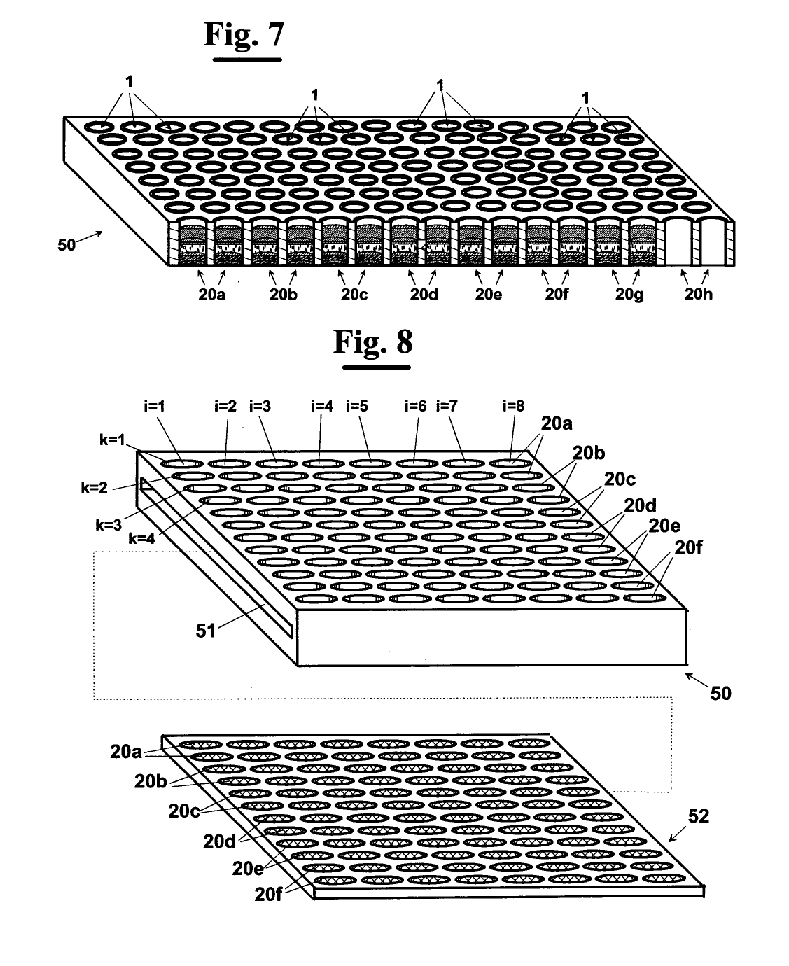

The different resulting solutions thus obtained pass into zone 3 of each container 1 and have each a specific group of proteins. In particular, two containers 1a are obtained containing acid proteins, two containers 1b are obtained containing basic proteins, two containers 1c are obtained containing hydrophobic proteins, two containers 1d are obtained containing phosphorylated proteins, two containers 1e are obtained containing glycosylated proteins, or alternatively, containers 1f are obtained containing proteins similar to a given antibody and containers 1f containing proteins similar to a given aptamer (FIG. 7).

From zone 3 of each container 1 the different resulting solutions are drawn and sent to an analysis instrument in order to check the different proteins of each unit isolated as above described. In order to make a comparison, two containers 1h are provided in which a determined amount is put of the complex mixture of proteins before sending the resulting solutions to the analytic instrument.

The isolated groups of proteins can be collected from the containers 1, for example at the respective bases, through a plurality of ducts 55.

As above described, to the sample to analyse a measured amount is added of a known specific proteolytic enzyme, for example trypsin, capable of breaking selectively some peptide bonds which are sent in series to a chromatographic column, for example of the high resolution type (HPLC), or to a plurality of columns, and, then transferred to a second instrument of analysis such as a mass spectrometer of MS type, or a mass spectrometer of MS/MS type.

Before putting the samples into containers 1 they can be preliminarily filtered for separating the mixture of proteins from other substances present in the sample of organic fluid so that the latter can removed.

To assist the operations of changing and of washing separation elements 20, plate 50 can be equipped with a housing 51 in which an insert 52 is put that is adapted to receive a determined number of separation elements 20.

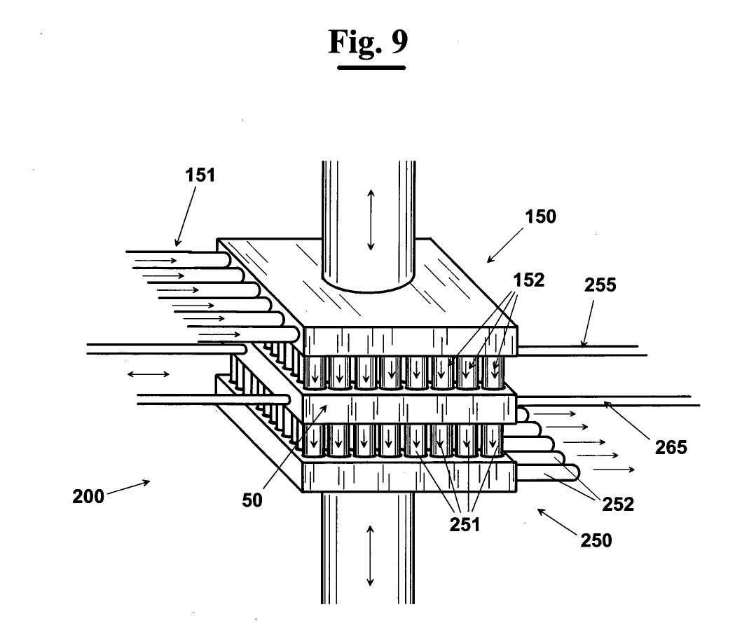

All the steps above described can be made in quick succession for example by an apparatus 200 shown in FIG. 9. It comprises a support plate 50 equipped with a determined number of containers 1 each of which has a separation element 20 as previously described in detail. Plate 50 can be slidingly mounted on rails 255 and 265, in order to be guided at a plurality of stations at which the steps above described are carried out.

In particular, an introduction plate 150 can be provided comprising a plurality of ducts 151 selectively connected with sample containers, not shown, which contain the samples to examine, or with solvent containers for the different solvents that can be used for separating the proteins at the respective separation elements 20. The introduction plate comprises, furthermore, a plurality of ducts 252 through which the different substances are put into different containers 1 at the respective upper apertures 2. Opposite to introduction plate 150 a collection plate 250 operates, which is also equipped with a plurality of collection ducts 251 through which the resulting solutions are collected from containers 1 at their lower aperture 3 and sent to the successive steps by means of ducts 252.

The foregoing description of a specific embodiment will so fully reveal the invention according to the conceptual point of view, so that others, by applying current knowledge, will be able to modify and/or adapt for various applications such an embodiment without further research and without parting from the invention, and it is therefore to be understood that such adaptations and modifications will have to be considered as equivalent to the specific embodiment. The means and the materials to realise the different functions described herein could have a different nature without, for this reason, departing from the field of the invention. It is to be understood that the phraseology or terminology employed herein is for the purpose of description and not of limitation.

1. Apparatus for separating the constituents of at least one complex mixture of proteins to submit to proteomic analysis, characterised in that it comprises:
a plurality of separation elements, said separation elements of said plurality having chemical-physical features such that they can capture selectively by adsorption proteins belonging to a determined homogeneous group of proteins of said complex mixture;
a holding means, for holding said plurality of separation elements according to a determined array,
a distribution means, said distribution means adapted to distribute said complex mixture of proteins on each separation element of said plurality of separation elements;
an application means, said application means adapted to apply on each separation element a determined solvent that can cause the removal of a respective group of proteins from the respective separation element obtaining respective resulting solutions;
a collection means, for collecting said respective resulting solutions for a following analysis in order to identify the single proteins of each group.

2. Apparatus for separating the constituents of a complex mixture of proteins, according to claim 1, wherein each separation element of said plurality of separation elements is a packed column of a packing material, said packing material permeated, in use, by the complex mixture of proteins and by said solvent.

3. Apparatus for separating the constituents of a complex mixture of proteins, according to claim 1, wherein said packing material is selected from the group comprised of:
chemically deactivated silica;
a porous polymer;
a nonporous polymer.

4. Apparatus for separating the constituents of a complex mixture of proteins, according to claim 1, wherein said means for collecting comprises at least one plate having microwells, or “microwell plate”.

5. Apparatus for separating the constituents of a complex mixture of proteins, according to claim 1, wherein said distribution means and/or said application means comprise multichannel pipettes.

6. Apparatus for separating the constituents of a complex mixture of proteins, according to claim 1, wherein said distribution means is operatively connected to a program means, said program means adapted to operate said distribution means so that they distribute a determined amount of said complex mixture into determined separation elements.

7. Apparatus for separating the constituents of a complex mixture of proteins, according to claim 1, wherein said application means is operatively connected to a program means, said program means adapted to operate said application means so that they apply determined amount of solvent into determined separation elements.

8. Apparatus for separating the constituents of a complex mixture of proteins, according to claim 1, wherein said separation elements comprise at least two of the following separation elements:
a strong anionic exchange separation element (SAX) that is adapted to separate acid proteins;
a strong cationic exchange separation element (SCX) that is adapted to separate basic proteins;
a reverse-phase separation element (RP-18) that is adapted to separate hydrophobic proteins;
a separation element based on chelant metals (IMAC) or on titanium dioxide or on other substances adapted to separate phosphorylated proteins;
a separation element based on Lectin, or Concanavalin-A, that is adapted to separate glycosylated proteins;
a separation element based on an antibody having specific affinity with a determined group of proteins, said separation element adapted to separate said group of proteins having said specific affinity with said antibody;
a separation element based on an aptamer having specific affinity with a determined group of proteins, said separation element adapted to separate said group of proteins having said specific affinity with said aptamer;
or a combination thereof.

9. Apparatus for separating the constituents of a complex mixture of proteins, according to claim 1, wherein each said separation element captures by adsorption proteins belonging to a homogeneous group of proteins of particular analytic use, said homogeneous group of proteins selected from the group comprised of:
a group of acid proteins,
a group of basic proteins,
a group of hydrophobic proteins,
a group of phosphorylated proteins,
a group of glycosylated proteins,
a group of proteins having specific affinity with a determined antibody;
a group of proteins having specific affinity with a determined aptamer.

10. Apparatus for separating the constituents of a complex mixture of proteins, according to claim 1, wherein said means for collecting comprises containers for said resulting solutions which consist of at least one among:
a container for acid proteins;
a container for basic proteins;
a container for hydrophobic proteins;
a container for phosphorylated proteins;
a container for glycosylated proteins;
a container for a homogeneous group of proteins having specific affinity with a determined antibody;
a container for a homogeneous group of proteins having specific affinity with a determined aptamer;
or a combination thereof.

11. Apparatus for separating the constituents of a complex mixture of proteins, according to claim 1, wherein, furthermore, a means is provided for collecting the non-adsorbed proteins on the corresponding separation element.

12. Apparatus for separating the constituents of a complex mixture of proteins, according to claim 1, wherein each said separation element comprises a first zone, or inlet zone, and a second zone, or outlet zone, said distribution means and said application means acting at said first zone, and said means for collecting the non-adsorbed proteins acting, instead, at said second zone.

13. Apparatus for separating the constituents of a complex mixture of proteins, according to claim 1, wherein said plurality of separation elements comprises at least a first and a second separation element that is adapted to separate by adsorption a same group of proteins.

14. Apparatus for separating the constituents of a complex mixture of proteins, according to claim 1, wherein said solvent is a buffer solution containing a desorbing agent that is adapted to induce at least one of the following effects on said complex mixture of proteins:
changing pH,
changing ionic force,
changing polarity.

15. Apparatus for separating the constituents of a complex mixture of proteins, according to claim 1, wherein said solvent is a buffer solution containing a soluble desorbing agent that is capable of competing with the adsorbing elements of the packing material of the separation element.

16. Apparatus for separating the constituents of a complex mixture of proteins, according to claim 1, wherein said solvent is a buffer solution containing a denaturing agent.

17. Apparatus for separating the constituents of a complex mixture of proteins, according to claim 1, wherein a means is provided for analysing said respective resulting solutions, said means for analysing adapted to identify the single proteins of each group.

18. Apparatus for separating the constituents of a complex mixture of proteins, according to claim 17, wherein said means for analysing said resulting solutions comprises a chromatograph for liquid chromatography at high resolution (HPLC) and a mass spectrometer, in particular said mass spectrometer selected from the group comprised of: a mass spectrometer of MS type, or a mass spectrometer of MS/MS type.

19. Method for separating the constituents of a complex mixture of proteins for proteomic analysis characterised in that it comprises the steps of:
arranging a plurality of separation elements according to a determined array, said separation elements adapted to separate selectively by adsorption a homogeneous group of proteins different from other proteins present in the complex mixture of proteins;
distributing at least one complex mixture of proteins on each separation element of said plurality of separation elements;
selectively separating by each separation element a determined group of proteins from the other proteins present in the complex mixture;
applying a determined solvent on each separation element, said solvent adapted to remove each group of proteins from the respective separation element obtaining respective resulting solutions;
collecting said resulting solutions from each separation element;
analysing the resulting solutions to identify the single proteins of each group.

20. Method for separating the constituents of a complex mixture of proteins, according to claim 19, wherein before said step of applying said determined solvent on said separation element a step is provided of washing each separation element, said washing step adapted to remove possible interfering molecules co-adsorbed on said separation element with each group of proteins.

21. Method for separating the constituents of a complex mixture of proteins, according to claim 19, wherein a preliminary step is provided of filtering a sample of organic fluid comprising said mixture of proteins, said filtering step adapted to separate said mixture of proteins from other substances which are present in the fluid sample and which are removed.

22. Method for separating the constituents of a complex mixture of proteins, according to claim 19, wherein before said step of analysing a dilution and neutralization of said resulting solutions is effected.
